(19) United States
(12) Patent Application Publication
(10) Pub. No.: US 2010/0249192 A1
(43) Pub. Date: Sep. 30, 2010

(54) NOVEL HETEROAROMATIC COMPOUNDS AS INHIBITORS OF STEAROYL-COENZYME A DELTA-9 DESATURASE

(75) Inventors: Chun Sing Li, Dollar-des-Ormeaux (CA); Yeeman K. Ramtohul, Pierrefonds (CA); Jean-Philippe Leclerc, Laval (CA)

Correspondence Address: MERCK, P O BOX 2000, RAHWAY, NJ, 07065-0907, US

(73) Assignee: MERCK FROSST CANADA LTD.

(21) Appl. No.: 12/744,062

(22) PCT Filed: Dec. 9, 2008
(86) PCT No.: PCT/CA08/02156

§ 371 Date: May 21, 2010

Related U.S. Application Data
(60) Provisional application No. 61/007,233, filed on Dec. 11, 2007.

(51) Int. Cl. A61K 31/4439; C07D 413/04; A61K 31/422; A61P 3/10; A61P 3/04; A61P 1/16; A61P 3/06; A61P 9/10; C07D 413/14; C07D 403/04; A61K 31/4192; A61K 31/41

(52) U.S. Cl. 514/340; 548/247; 514/378; 546/272.1; 548/253; 514/381; 514/382; 548/236; 514/374

(57) ABSTRACT

Heteroaromatic compounds of structural formula I are inhibitors of stearoyl-coenzyme A delta-9 desaturase (SCD). The compounds of the present invention are useful for the prevention and treatment of conditions related to abnormal lipid synthesis and metabolism, including cardiovascular disease; atherosclerosis; obesity; diabetes; neurological disease; Metabolic Syndrome; insulin resistance; cancer; liver steatosis; and non-alcoholic steatohepatitis.

HetAr⁻W⁻X⁻Ar (I)

### FIELD OF THE INVENTION

The present invention relates to novel heteroaromatic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease, such as atherosclerosis; obesity; diabetes; neurological disease; metabolic syndrome; insulin resistance; cancer; and hepatic steatosis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCDs) catalyze the rate-limiting formation of the cis-double bond at the C₉-C₁₀ position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCDs in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD1 gene (Zheng et al., Nature Genetics, 23: 268-270 (1999)), b) SCD1-null mice from targeted gene deletion (Ntambi, et al., PNAS, 99: 11482-11486 (2002), and c) the suppression of SCD1 expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. Thus, SCD inhibition represents a novel therapeutic strategy in the treatment of obesity and related metabolic disorders.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). Elevated SCD activity in adipose tissue is closely coupled to the development of insulin resistance (Sjogren, et al., Diabetologia, 51(2): 328-35 (2007)). The postprandial de novo lipogenesis is significantly elevated in obese subjects (Marques-Lopes, et al., American Journal of Clinical Nutrition, 73: 252-261 (2001)). Knockout of the SCD gene ameliorates Metabolic Syndrome by reducing plasma triglycerides, reducing weight gain, increasing insulin sensitivity, and reduces hepatic lipid accumulation (MacDonald, et al., Journal of Lipid Research, 49(1): 217-29 (2007)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)). RNA interference of SCD-1 reduces human tumor cell survival (Morgan-Lappe, et al., Cancer Research, 67(9): 4390-4398 (2007)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications WO 2005/011653, WO 2005/011654, WO 2005/011656, WO 2005/011656, WO 2005/011657, WO 2006/014168, WO 2006/034279, WO 2006/034312, WO 2006/034315, WO 2006/034338, WO 2006/034341, WO 2006/034440, WO 2006/034441, WO 2006/034446, WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; WO 2007/136746; and WO 2008/074835, all assigned to Xenon Pharmaceuticals, Inc.

A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/056846 (24 May 2007); WO 2007/071023 (28 Jun. 2007); WO 2007/134457 (29 Nov. 2007); WO 2007/143823 (21 Dec. 2007); WO 2007/143824 (21 Dec. 2007); WO 2008/017161 (14 Feb. 2008); WO 2008/046226 (24 Apr. 2008); WO 2008/064474 (5 Jun. 2008); and US 2008/0182838 (31 Jul. 2008).

WO 2008/003753 (assigned to Novartis) discloses a series of pyrazolo[1,5-a]pyrimidine analogs as SCD inhibitors; WO 2007/143597 and WO 2008/024390 (assigned to Novartis and Xenon Pharmaceuticals) disclose heterocyclic derivatives as SCD inhibitors; and WO 2008/096746 (assigned to Takeda Pharmaceutical) disclose spiro compounds as SCD inhibitors.

Small molecule SCD inhibitors have also been described by (a) G. Liu, et al., “Discovery of Potent, Selective, Orally Bioavailable SCD1 Inhibitors,” in J. Med. Chem., 50: 3086-3100 (2007); (b) H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007); and (c) Z. Xin, et al., “Discovery of piperidine-aryl urea-based stearoyl-CoA desaturase 1 inhibitors,” Bioorg. Med. Chem. Lett., 18: 4298-4302 (2008).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, diabetes, metabolic syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobrzyn and J. M. Ntambi, in “Stearoyl-CoA desaturase as a new drug target for obesity treatment,” Obesity Reviews, 6: 169-174 (2005).

### SUMMARY OF THE INVENTION

The present invention relates to heteroaromatic compounds of structural formula

HetAr—W—X—Ar (I)

These heteroaromatic compounds are effective as inhibitors of SCD. They are therefore useful for the treatment, control or prevention of disorders responsive to the inhibition of SCD, such as diabetes, insulin resistance, lipid disorders, obesity, atherosclerosis, and metabolic syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, and metabolic syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with novel heteroaromatic compounds useful as inhibitors of SCD. Compounds of the present invention are described by structural formula I:

HetAr—W—X—Ar (I)

and pharmaceutically acceptable salt thereof; wherein

### X is —O—, —S—, —S(O)—, —S(O)₂—, —NR⁹—, or —CR¹⁰R¹¹—;

W is selected from the group consisting of:

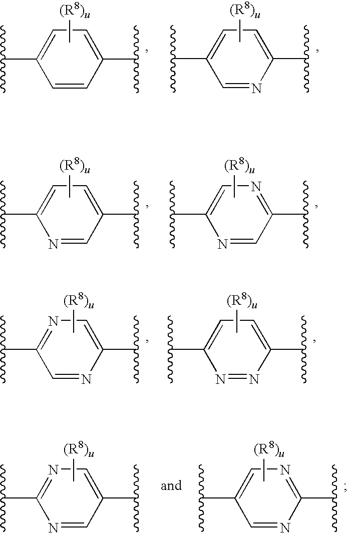

HetAr is heteroaryl selected from the group consisting of:

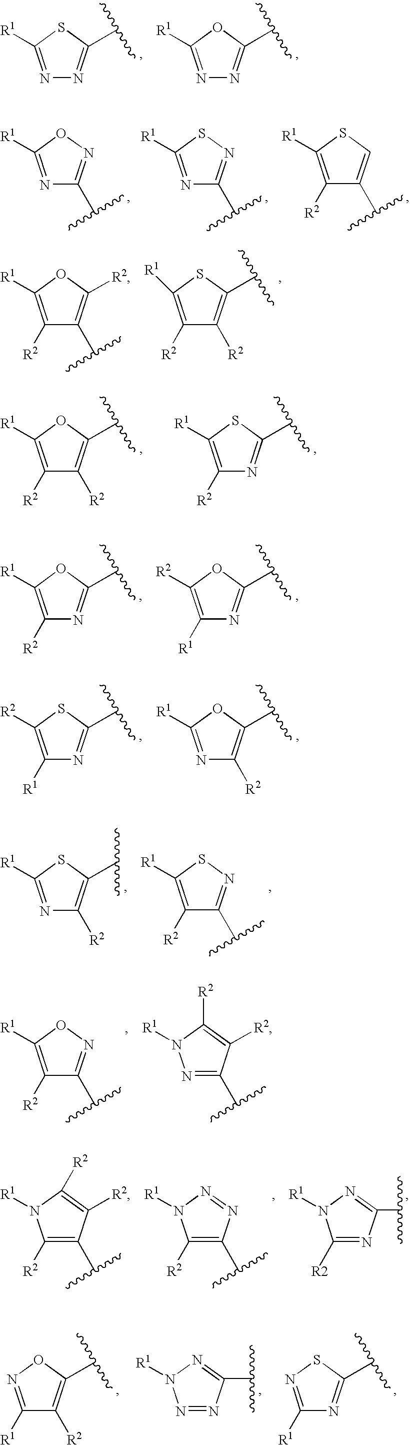

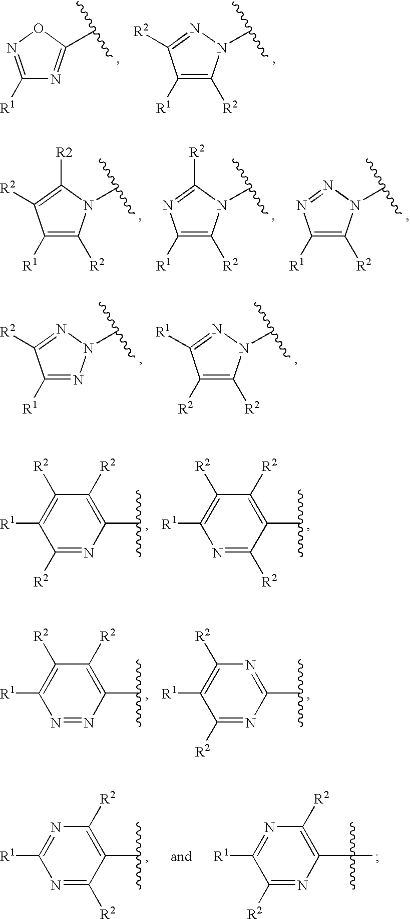

R¹ is heteroaryl selected from the group consisting of:

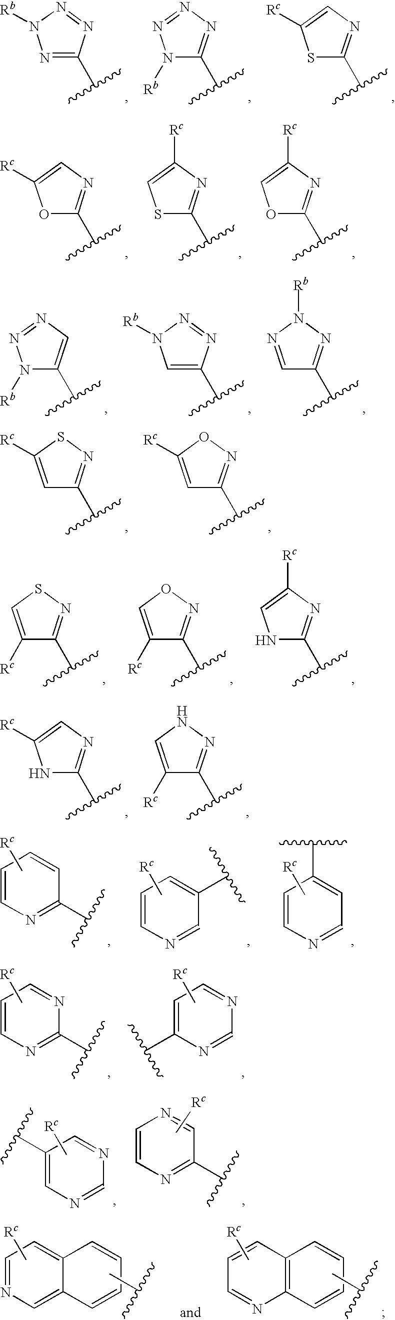

wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;
and wherein said R¹ heteroaryl ring is optionally substituted with a substituent selected from the group consisting of cyano, halogen, C₁₋₄ alkyl, C₁₋₄ alkoxy, C₁₋₄ alkylthio, C₁₋₄ alkylsulfonyl, and trifluoromethyl;
each R² is independently selected from the group consisting of:

hydrogen,

halogen,

hydroxy,

cyano,

amino,

nitro,

C₁₋₄ alkyl, optionally substituted with one to five fluorines,

C₁₋₄ alkoxy, optionally substituted with one to five fluorines,

C₁₋₄ alkylthio, optionally substituted with one to five fluorines, P C₁₋₄ alkylsulfonyl,

carboxy,

C₁₋₄ alkyloxycarbonyl, and

C₁₋₄ alkylcarbonyl;

Ar is phenyl or naphthyl optionally substituted with one to five R³ substituents;
each R³ is independently selected from the group consisting of:

C₁₋₆ alkyl,

C₂₋₆ alkenyl,

(CH₂)ₙ-phenyl,

(CH₂)ₙ-naphthyl,

(CH₂)ₙ-heteroaryl,

(CH₂)ₙ-heterocyclyl,

(CH₂)ₙC₃₋₇ cycloalkyl,

halogen,

nitro,

(CH₂)ₙOR⁴,

(CH₂)ₙN(R⁴)₂,

(CH₂)ₙC≡N,

(CH₂)ₙCO₂R⁴,

(CH₂)ₙNR⁴SO₂R⁴

(CH₂)ₙSO₂N(R⁴)₂,

(CH₂)ₙS(O)₀₋₂R⁴,

(CH₂)ₙNR⁴C(O)NR⁴)₂,

(CH₂)ₙC(O)N(R⁴)₂,

(CH₂)ₙNR⁴C(O)R⁴,

(CH₂)ₙNR⁴CO₂R⁴,

(CH₂)ₙC(O)R⁴,

O(CH₂)ₙC(O)N(R⁴)₂,

(CH₂)ₛ—Z—(CH₂)ₜ-phenyl,

(CH₂)ₛ—Z—(CH₂)ₜ-naphthyl,

(CH₂)ₛ—Z—(CH₂)ₜ-heteroaryl,

(CH₂)ₛ—Z—(CH₂)ₜ-heterocyclyl,

(CH₂)ₛ—Z—(CH₂)ₜ—C₃₋₇ cycloalkyl,

(CH₂)ₛ—Z—(CH₂)ₜ—OR⁴,

(CH₂)ₛ—Z—(CH₂)ₜ₋N(R⁴)₂,

(CH₂)ₛ—Z—(CH₂)ₜ—NR⁴SO₂R⁴,

(CH₂)ₛ—Z—(CH₂)ₜ—C≡N,

(CH₂)ₛ—Z—(CH₂)ₜ—CO₂R⁴,

(CH₂)ₛ—Z—(CH₂)ₜ—SO₂N(R⁴)₂,

(CH₂)ₛ—Z—(CH₂)ₜ—S(O)₀₋₂R⁴,

(CH₂)ₛ—Z—(CH₂)ₜ₋NR⁴C(O)N(R⁴)₂,

(CH₂)ₛ—Z—(CH₂)ₜ—C(O)N(R⁴)₂,

(CH₂)ₛ—Z—(CH₂)ₜ—NR⁴C(O)R⁴,

(CH₂)ₛ—Z—(CH₂)ₜ—NR⁴CO₂R⁴,

(CH₂)ₛ—Z—(CH₂)ₜ—C(O)R⁴,

CF₃,

CH₂CF₃,

OCF₃, and

OCH₂CF₃;

in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;

### Z is O, S, or NR⁴;

each R⁴ is independently selected from the group consisting of

hydrogen,

C₁₋₆ alkyl,

(CH₂)ₙ-phenyl,

(CH₂)ₙ-heteroaryl,

(CH₂)ₙ-naphthyl, and

(CH₂)ₙC₃₋₇ cycloalkyl;

wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
each R⁶ and R⁷ are independently hydrogen or C₁₋₃ alkyl, wherein alkyl is optionally substituted with one to five fluorines;
each R⁸ is independently selected from the group consisting of hydrogen, halogen, and C₁₋₄ alkyl wherein alkyl is optionally substituted with one to five fluorines;
R⁹, R¹⁰, and R¹¹ are each independently hydrogen or C₁₋₃ alkyl, wherein alkyl is optionally substituted with one to five fluorines;
u is an integer from 0 to 2;
r is an integer from 0 to 3;
m is an integer from 1 to 3;
each p is independently an integer from 1 to 3;
each n is independently an integer from 0 to 2;
each s is independently an integer from 1 to 3; and
each t is independently an integer from 1 to 3.

In one embodiment of the compounds of the present invention, X is O.

In a second embodiment of the compounds of the present invention, Ar is phenyl substituted with one to three R³ substituents as defined above.

In a third embodiment of the compounds of the present invention, W is phenyl or pyridyl wherein phenyl and pyridyl are optionally substituted with one or two R⁸ substituents as defined above. In a class of this embodiment, W is unsubstituted phenyl.

In a fourth embodiment of the compounds of the present invention, HetAr is heteroaryl selected from the group consisting of:

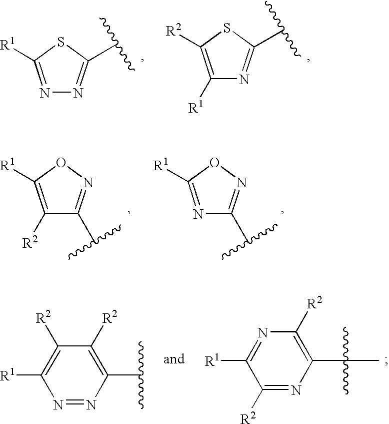

wherein R¹ and R² are as defined above. In a class of this embodiment, R² is hydrogen. In another class of this embodiment, HetAr is

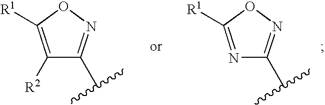

wherein R¹ and R² are as defined above. In a subclass of this class, R² is hydrogen. In another subclass of this class, HetAr is

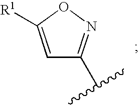

wherein R¹ is as defined above.

In a fifth embodiment of the compounds of the present invention, R¹ is heteroaryl selected from the group consisting of:

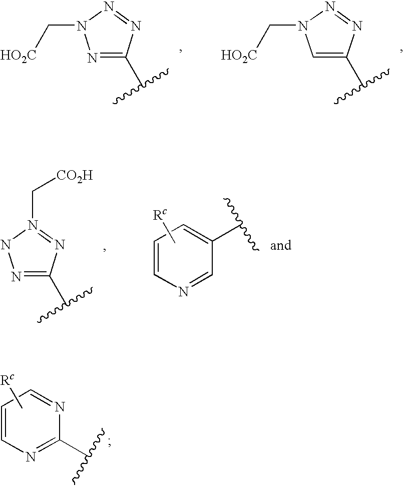

wherein R^{c} is —CO₂H, —CO₂C₁₋₃ alkyl, —CH₂CO₂H, or —CH₂CO₂C₁₋₃ alkyl. In a class of this embodiment, R¹ is

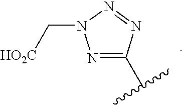

In a sixth embodiment of the compounds of the present invention, HetAr is heteroaryl selected from the group consisting of:

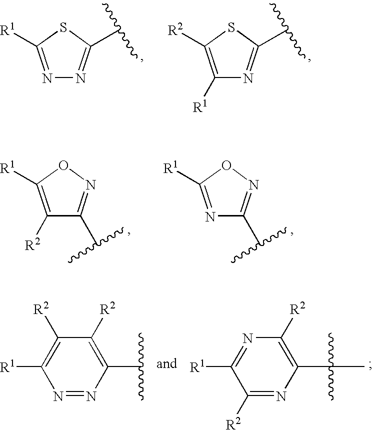

and R¹ is heteroaryl selected from the group consisting of:

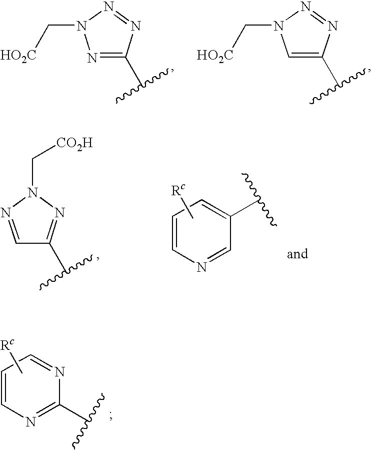

wherein R^{c} is —CO₂H, —CO₂C₁₋₃ alkyl, —CH₂CO₂H, or —CH₂CO₂C₁₋₃ alkyl.

In a class of this embodiment, HetAr is

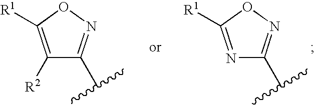

### and R¹ is

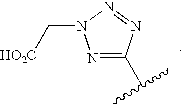

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. When no number of carbon atoms is specified, C₁₋₆ is intended.

“Cycloalkyl” means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkenyl” shall mean straight or branched-chain alkenes having the specified number of carbon atoms. Examples of alkenyl include vinyl, 1-propenyl, 1-butenyl, 2-butenyl, and the like.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, and 2-oxoazetidin-1-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group conisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, and other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD-Induced Rat Liver Microsome Assay:

The activity of compounds of formula I against the SCD enzyme was determined by following the conversion of radiolabeled-stearoyl-CoA to oleoyl-CoA using SCD-induced rat liver microsome and a previously published procedure with some modifications (Joshi, et al., J. Lipid Res., 18: 32-36 (1977)). After feeding wistar rats with a high carbohydrate/fat-free rodent diet (LabDiet # 5803, Purina) for 3 days, the SCD-induced livers were homogenized (1:10 w/v) in 250 mM sucrose, 1 mM EDTA, 5 mM DTT and 50 mM Tris-HCl (pH 7.5). After a 20 min centrifugation (18,000×g/4° C.) to remove tissue and cell debris, the microsome was prepared by a 100,000×g centrifugation (60 min) with the resulting pellet suspended in 100 mM sodium phosphate, 20% glycerol and 2 mM DTT. Test compound in 2 μL DMSO was incubated for 15 min at room temperature with 180 μL of the microsome (typically at about 100 μg/mL, in Tris-HCl buffer (100 mM, pH 7.5), ATP (5 mM), Coenzyme A (0.1 mM), Triton X-100 (0.5 mM) and NADH (2 mM)). The reaction was initiated by the addition of 20 μL of [³H]-Stearoyl-CoA (final concentration at 2 μM with the radioactivity concentration at 1 μCi/mL), and terminated by the addition of 150 μL, of 1N sodium hydroxide. After 60 min at room temperature to hydrolyze the oleoyl-CoA and stearoyl-CoA, the solution was acidified by the addition of 150 μL of 15% phosphoric acid (v/v) in ethanol supplemented with 0.5 mg/mL stearic acid and 0.5 mg/mL oleic acid. [³H]-oleic acid and [³H]-stearic acid were then quantified on a HPLC that is equipped with a C-18 reverse phase column and a Packard Flow Scintillation Analyzer. Alternatively, the reaction mixture (80 μL) was mixed with a calcium chloride/charcoal aqueous suspension (100 μL of 15% (w/v) charcoal plus 20 μL of 2 N CaCl₂). The resulting mixture was centrifuged to precipitate the radioactive fatty acid species into a stable pellet. Tritiated water from SCD-catalyzed desaturation of 9,10-[³H]-stearoyl-CoA was quantified by counting 50 μL of the supernant on a scintillation counter.

### II. Whole Cell-Based SCD (Delta-9), Delta-5 and Delta-6 Desaturase Assays:

Human HepG2 cells were grown on 24-well plates in MEM media (Gibco cat#11095-072) supplemented with 10% heat-inactivated fetal bovine serum at 37° C. under 5% CO₂ in a humidified incubator. Test compound dissolved in the media was incubated with the subconfluent cells for 15 min at 37° C. [1-¹⁴C]-stearic acid was added to each well to a final concentration of 0.05 μCi/mL to detect SCD1-catalyzed [¹⁴C]-oleic acid formation. 0.05 μCi/mL of [1-¹⁴C]-eicosatrienoic acid or [1-14C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 of acetonitrile and quantified on a HPLC that was equipped with a C-18 reverse phase column and a Packard Flow Scintillation Analyzer. The levels of [¹⁴C]-oleic acid over [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD1, delta-5 and delta-6 desaturase, respectively.

The SCD inhibitors of formula I, particularly the inhibitors of Examples 1 to 13, exhibit an inhibition constant IC₅₀ of less than 1 μM and more typically less than 0.1 μM. Generally, the IC₅₀ ratio for delta-5 or delta-6 desaturases to SCD for a compound of formula I, particularly for Examples 1 to 13, is at least about ten or more, and preferably about one hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-14C]-stearic acid, was dosed at 20 μCi/kg IV. At 3 h post dosing of the compound, the liver was harvested and then hydrolyzed in 10 N sodium hydroxide for 24 h at 80° C., to obtain the total liver fatty acid pool. After phosphoric acid acidification of the extract, the amount of [1-¹⁴C]-stearic acid and [1-¹⁴C]-oleic acid was quantified on a HPLC that was equipped with a C-18 reverse phase column and a Packard Flow Scintillation Analyzer.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase-IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) agonists of GPR-119.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (MK-0431); vildagliptin (LAF 237); denagliptin; P93/01; saxagliptin (BMS 477118); RO0730699; MP513; SYR-322: ABT-279; PHX1149; GRC-8200; and TS021.

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:

(1) a compound of structural formula I;
(2) a compound selected from the group consisting of:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors

(lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) agonists of GPR-119; and

(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### List of Abbreviations:

Alk=alkyl
APCI=atmospheric pressure chemical ionization
Ar=aryl
Boc=tert-butoxycarbonyl
br=broad
t-BuONO=t-butyl nitrite
d=doublet
DBU=1,8-diazabicyclo[5.4.0]undec-7-ene

### DMF=N,N-dimethylformamide

DIBAL-H=diisobutylaluminum hydride
DMSO=dimethyl sulfoxide
ESI=electrospray ionization
ESMS=electrospray ion-mass spectroscopy
EtOAc=ethyl acetate
HPLC=high-performance liquid chromatography
m=multiplet
min=minutes
MeOH=methyl alcohol
MS=mass spectroscopy
NaHMDS=sodium bis(trimethylsilyl)amide
NMP=1-methyl-2-pyrrolidinone
NMR=nuclear magnetic resonance spectroscopy
PG=protecting group
P=pentuplet
Q=quartet
rt=room temperature
s=singlet
t=triplet
TFAA=trifluoroacetic anhydride
Tf₂O=trifluoromethanesulfonic anhydride
THF=tetrahydrofuran
TLC=thin-layer chromatography
TsOH=toluene-4-sulfonic acid

### Preparation of Compounds of the Invention:

The compounds of structural formula I can be prepared according to the procedures of the following Scheme and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESMS).

### Method A (Scheme 1):

An appropriately substituted halo-arylcarboxaldehyde or halo-heteroarylcarboxaldehyde 1 is reacted with a nucleophile 2 in the presence of a base, such as alkali metal (K, Na, Cs) carbonate, in a solvent such as DMF at a temperature range of from room temperature to refluxing temperature to give 3. Treatment of 3 with hydroxylamine hydrochloride in a solvent, such as THF/water and EtOH/water, in the presence of a base, such as sodium carbonate, gives oxime 4. Oxime 4 is converted into the imidoyl chloride intermediate 5 by the treatment of 4 with N-chlorosuccinimide (NCS) in a solvent such as DMF. Subsequent cyclization with an ester of propiolic acid, such as methyl propiolate, in the presence of a base such as triethylamine in a solvent such as DMF gives isoxazole 6. Treatment of 6 with ammonium hydroxide in a solvent, such as methanol and THF, provides amide 7. Dehydration of 7 with trifluoroacetic anhydride (TFAA) in the presence of a base such as triethylamine affords nitrile 8. Nitrile 8 is then reacted with NaN₃ in the presence of a Lewis acid such as NH₄Cl in a solvent such as DMF to give tetrazole 9. Alkylation of 9 with an haloalkyl ester, such as ethyl bromoacetate, in the presence of a base, such as triethylamine, Cs₂CO₃, K₂CO₃, and t-BuOK, in a solvent such as DMF usually gives a mixture of 1-alkylated and 2-alkylated isomers. The 2-alkylated isomer 10 is usually the major isomer and can be separated from the minor 1-alkylated isomer by chromatographic methods, such as flash column chromatography. Hydrolysis of the ester group in 10 with an alkaline base such as NaOH in a solvent such as THF with an alcoholic solvent such as MeOH provides the desired carboxylic acid final product 11.

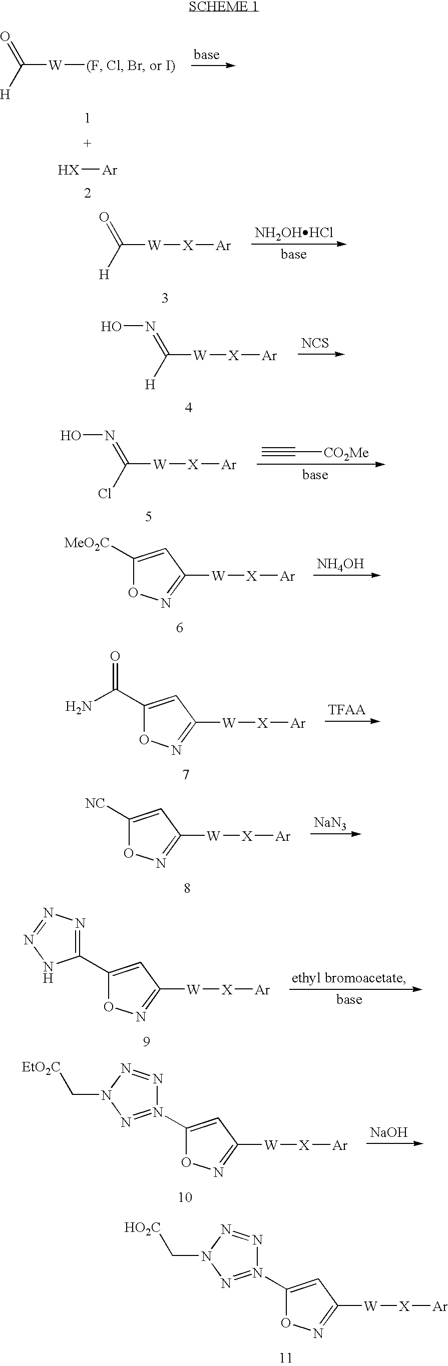

### Method B (Scheme 2):

An appropriately substituted halo-arylnitrile or halo-heteroarylnitrile 12 is reacted with a nucleophile 2 in the presence of a base, such as alkali metal (K, Na, Cs) carbonate, in a solvent such as DMF at a temperature range of from room temperature to refluxing temperature to give 13. Reaction of 13 with hydroxylamine hydrochloride in a solvent such as EtOH in the presence of a base such as triethylamine under refluxing condition gives the carboximidamide 14. Reaction of carboximidamide 14 with methyl oxalyl chloride in the presence of a base, such as triethylamine and sodium hydride, in a solvent, such as THF, at room temperature or under refluxing conditions affords the methyl oxadiazole-5-carboxylate intermediate which upon reaction with ammonia affords the oxadiazole-5-carboxamide 15. Dehydration of 15 with trifluoroacetic anhydride (TFAA) in the presence of a base such as triethylamine affords nitrile 16. Nitrile 16 is then reacted with NaN₃ in the presence of a Lewis acid such as NH₄Cl in a solvent such as DMF to give tetrazole 17. Alkylation of 17 with a haloalkyl ester, such as ethyl bromoacetate, in the presence of a base, such as triethylamine, Cs₂CO₃, K₂CO₃, and t-BuOK, in a solvent such as DMF usually gives a mixture of 1-alkylated and 2-alkylated isomers. The 2-alkylated isomer 18 is usually the major isomer and can be separated from the minor 1-alkylated isomer by chromatographic methods, such as flash column chromatography, followed by recrystallization or trituration with a solvent such as Et₂O. Hydrolysis of the ester group in 18 with an alkaline base such as NaOH in a solvent such as THF with an alcoholic solvent such as MeOH provides the carboxylic acid final product 19.

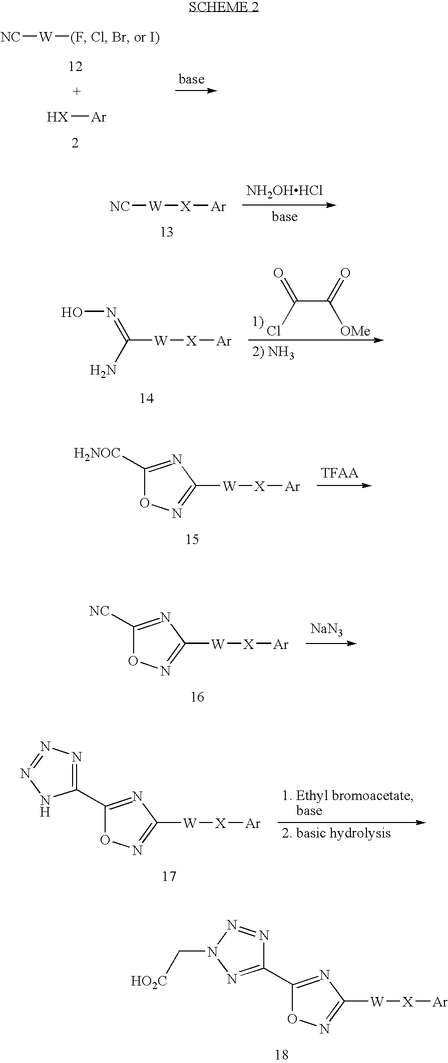

### Method C (Scheme 3):

An appropriately substituted aryl or heteroaryl halide 19 is coupled with a nitrogen heterocycle 20. The resulting product 21 is then converted into the desired target compound 25 in a similar manner as described for Methods A and B.

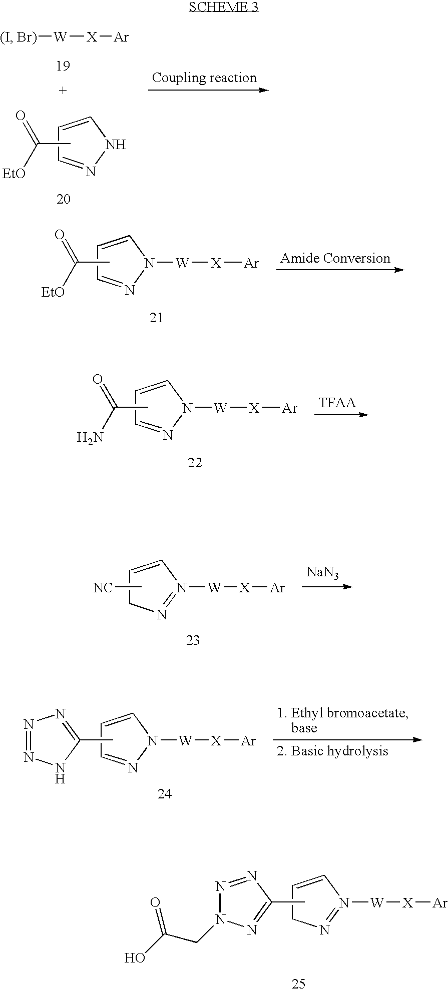

### Method D (Scheme 4):

An appropriately substituted aryl or heteroaryl azide 26 is reacted with a propiolate ester 27. The resulting cycloaddition product 28 is then converted into the target compound 32 in a similar manner as described for Methods A and B.

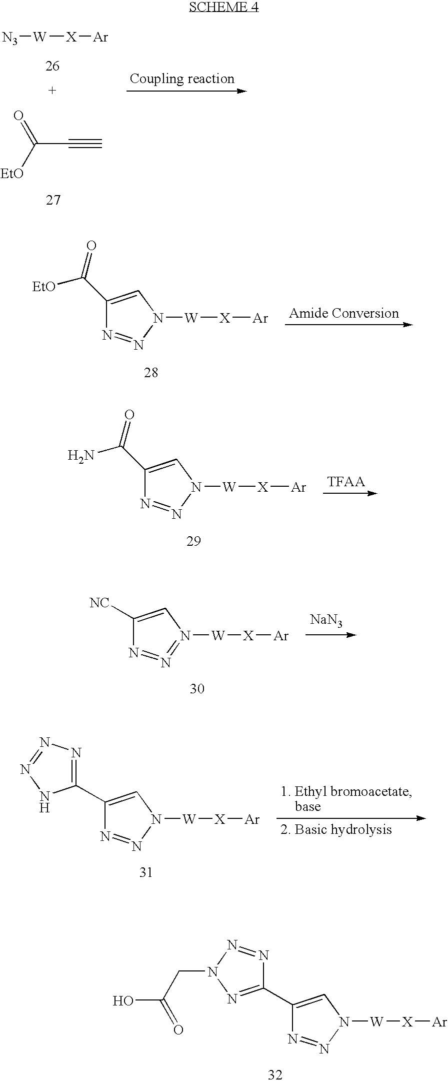

### Method E (Scheme 5):

An appropriately substituted boronate or boronic acid 33 is coupled with an appropriately substituted heterocyclic halide 34. The resulting product 35 is then converted into the target compound 38 in a similar manner as described for Methods A and B.

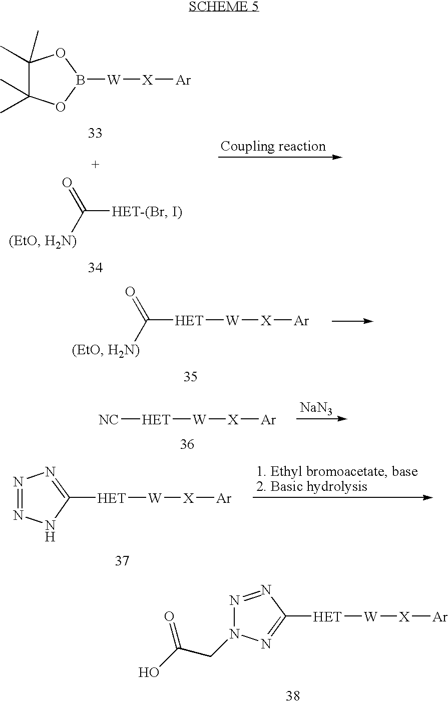

### Method F (Scheme 6):

An appropriately substituted halo-arylester or halo-heteroarylester 39 is reacted with a nucleophile 2 in the presence of a base, such as alkali metal (K, Na, Cs) carbonate, in a solvent such as DMF at a temperature range of from room temperature to refluxing temperature to provide intermediate 40, after hydrolysis of the ester group with an alkaline base such as NaOH in a solvent such as methanolic THF. Treatment of 40 with the serine methyl ester hydrochloride in a solvent, such as DMF, in the presence of a base, such as N-methylmorpholine, and a coupling reagent provides amide 41. Intramolecular cyclisation is performed using CCl₄, a phosphine, such as PPh₃, and a base, such as DIPEA, to generate heterocycle 42. Heterocycle 42 is then converted into oxazole 43 using a copper source, such as CuBr₂, and base, such as DBU. Treatment of 43 with ammonia gas in a solvent system such as methanol and THF provides amide 44. Dehydration of 44 with trifluoroacetic anhydride (TFAA) in the presence of a base such as triethylamine affords nitrile 45. Nitrile 45 is then reacted with NaN₃ in the presence of a Lewis acid such as NH₄Cl in a solvent such as DMF to give tetrazole 46. Alkylation of 46 with an haloalkyl ester, such as ethyl bromoacetate, in the presence of a base, such as triethylamine, Cs₂CO₃, K₂CO₃, and t-BuOK, in a solvent such as DMF usually gives a mixture of 1-alkylated and 2-alkylated isomers. The 2-alkylated isomer is usually the major isomer and can be separated from the minor 1-alkylated isomer by chromatographic methods, such as flash column chromatography. Hydrolysis of the ester group with an alkaline base such as NaOH in a solvent such as methanolic THF provides the desired carboxylic acid 47.

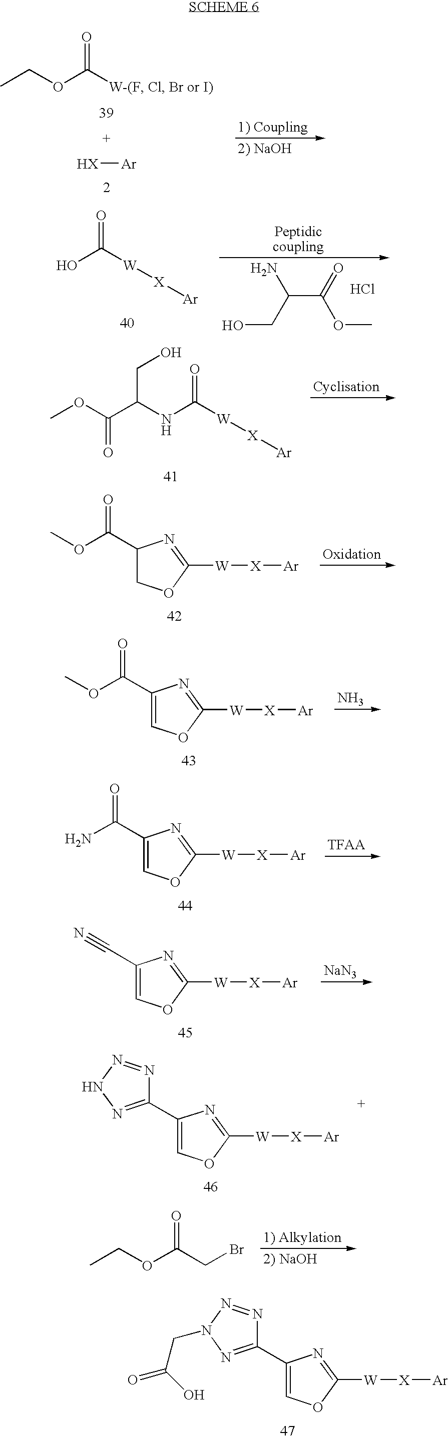

The following Examples are provided to illustrate the invention and are not to be construed as limiting the scope of the invention in any manner.

### Example 1

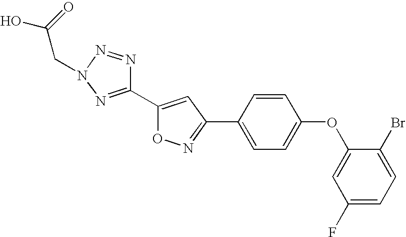

### (5-{3-[4-(2-Bromo-5-fluorophenoxy)phenyl]isoxazol-5-yl}-2H-tetrazol-2-yl)acetic acid

### Step 1: 4-(2-Bromo-5-fluorophenoxy)benzaldehyde

A mixture of 4-fluorobenzaldehyde (5 g, 40.3 mmol), 2-bromo-5-fluorophenol (8.08 g, 42.3 mmol) and Cs₂CO₃ (14.44 g, 44.3 mmol) in DMF (50 mL) was stirred at 100° C. overnight. After cooling, the mixture was diluted with water, acidified with 1 N HCl and extracted with EtOAc. The EtOAc extract was washed with dilute brine solution, dried (Na₂SO₄) and concentrated. Chromatography over silica gel and elution with hexanes:EtOAc (4:1) afforded the title compound as a light brown oil.

¹H NMR (500 MHz, acetone-d₆): δ 10.01 (s, 1H), 8.00 (d, 2H), 7.83 (dd, 1H), 7.18-7.10 (m, 4H).

### Step 2: 4-(2-Bromo-5-fluorophenoxy)benzaldehyde oxime

To a mixture of 4-(2-bromo-5-fluorophenoxy)benzaldehyde (3 g, 10.17 mmol) in THF (20 mL) and a solution of hydroxylamine hydrochloride (1.413 g, 20.33 mmol) in water (10 mL) was added a solution of 2 M sodium carbonate (10.17 ml, 20.33 mmol) at 0° C. The mixture was slowly warmed to rt and stirred overnight. Volatile materials were removed in vacuo. The residue was diluted with water and extracted with EtOAc. The EtOAc extract was washed with dilute brine solution, dried (Na₂SO₄) and concentrated to give the title compound as a light brown oil.

### Step 3: 4-(2-Bromo-5-fluorophenoxy)-N-hydroxybenzenecarboximidoyl chloride

To a solution of 4-(2-bromo-5-fluorophenoxy)benzaldehyde oxime (3 g, 9.67 mmol) in DMF (10 mL) was added portionwise N-chlorosuccinimide (1.4 g, 10.48 mmol) over about 15 min. The mixture was further stirred at rt for 1 h. After dilution with water, the mixture was extracted with EtOAc. The EtOAc extract was washed twice with water, dried (Na₂SO₄) and concentrated to give the crude title compound as a brown oil.

¹H NMR (500 MHz, acetone-d₆): δ 11.43 (s, 1H), 7.91 (d, 2H), 7.85-7.76 (m, 1 μl), 7.10 (d, 2H), 7.07-7.00 (m, 2H).

### Step 4: Methyl 3-[4-(2-bromo-5-fluorophenoxy)phenyl]isoxazole-5-carboxylate

To a solution of 4-(2-bromo-5-fluorophenoxy)-N-hydroxybenzenecarboximidoyl chloride (3.3 g, 9.58 mmol) and methyl propiolate (2.416 mL, 28.7 mmol) in DMF (30 mL) was added dropwise triethylamine (2.67 mL, 19.15 mmol) over a period of about 15 min. After further stirring for 2 h, the mixture was quenched with water, acidified with 1 N HCl and extracted with EtOAc. The EtOAc extract was washed three times with water, dried (Na₂SO₄) and concentrated. Chromatography over silica gel and elution with hexanes:EtOAc (4:1) provided a product which was triturated with hexanes:Et₂O (2:1) to give the title compound as a pale yellow solid.

¹H NMR (500 MHz, acetone-d₆): δ 8.05 (d, 2H), 7.81 (dd, 1H), 7.66 (s, 1H), 7.19 (d, 2H), 7.10-7.03 (m, 2H), 4.00 (s, 3H).

### Step 5: 3-[4-(2-Bromo-5-fluorophenoxy)phenyl]isoxazole-5-carboxamide

A mixture of methyl 3-[4-(2-bromo-5-fluorophenoxy)phenyl]isoxazole-5-carboxylate (1.5 g, 3.82 mmol) and ammonium hydroxide (10 mL, 71.9 mmol) in THF (10 mL) and MeOH (5 mL) was stirred at rt overnight. The mixture became homogenous after about 5-10 min and then a precipitate appeared. Volatile materials were removed in vacuo. The residue was suspended in water. The solid was collected, washed with water and Et₂O and dried under vacuum to give the title compound as a white solid.

¹H NMR (500 MHz, acetone-d₆): δ 8.02 (d, 2H), 7.81 (dd, 1H), 7.77 (s, 1H), 7.46 (s, 1H), 7.30 (s, 1H), 7.18 (d, 2H), 7.06 (m, 2H).

### Step 6: 3-[4-(2-Bromo-5-fluorophenoxy)phenyl]isoxazole-5-carbonitrile

To a suspension of 3-[4-(2-bromo-5-fluorophenoxy)phenyl]isoxazole-5-carboxamide (1.3 g, 3.45 mmol) and triethylamine (1.2 mL, 8.61 mmol) in CH₂Cl₂ (15 mL) at 0° C. was added TFAA (0.6 mL, 4.25 mmol). The cooling bath was then removed and the mixture was stirred at room temperature for 2 h. After quenching with saturated aqueous NaHCO₃, the mixture was extracted with CH₂Cl₂. The CH₂Cl₂ extract was washed with brine, dried (Na₂SO₄) and concentrated. Chromatography over silica gel and elution with hexanes:EtOAc (5:1) gave the title compound as a colorless oil which solidified on standing.

¹H NMR (500 MHz, acetone-d₆): δ 8.05-7.98 (m, 3H), 7.82 (t, 1H), 7.20 (d, 2H), 7.12-7.05 (m, 2H).

### Step 7: 5-{3-[4-(2-Bromo-5-fluorophenoxy)phenyl]isoxazol-5-yl}-1H-tetrazole

A mixture of 3-[4-(2-bromo-5-fluorophenoxy)phenyl]isoxazole-5-carbonitrile (1.1 g, 3.06 mmol), ammonium chloride (0.35 g, 6.54 mmol) and sodium azide in DMF (7 mL) was heated at 105° C. for 2 h. After cooling, the mixture was diluted with water, acidified with 1 M HCl and extracted with EtOAc. The EtOAc extract was washed with dilute brine solution, dried (Na₂SO₄) and concentrated to give the title compound.

¹H NMR (500 MHz, acetone-d₆): δ 8.11 (d, 2H), 7.84-7.79 (m, 1H), 7.75 (s, 1H), 7.21 (d, 2H), 7.12-7.05 (m, 2H).

### Step 8: Ethyl (5-{3-[4-(2-bromo-5-fluorophenoxy)phenyl]isoxazol-5-yl}-2H-tetrazol-2-yl)acetate

A mixture of 5-{3-[4-(2-bromo-5-fluorophenoxy)phenyl]isoxazol-5-yl}-1H-tetrazole (0.5 g, 1.243 mmol), ethyl bromoacetate (0.2 mL, 1.796 mmol) and triethylamine (0.4 mL, 2.87 mmol) in THF (20 mL) was refluxed for 2 h. After cooling, the mixture was diluted with water and extracted with EtOAc. The EtOAc extract was washed with water, dried (Na₂SO₄) and concentrated. Chromatography over silica gel and elution with hexanes:EtOAc (5:1) and trituration with hexanes:Et₂O (1:1) gave the title compound as a white solid (6:1 mixture of regioisomers).

### Step 9: (5-{3-[4-(2-Bromo-5-fluorophenoxy)phenyl]isoxazol-5-yl}-2H-tetrazol-2-yl)acetic acid

A mixture of ethyl (5-{3-[4-(2-bromo-5-fluorophenoxy)phenyl]isoxazol-5-yl}-2H-tetrazol-2-yl)acetate (500 mg, 1.024 mmol) and 1 N NaOH (2.1 mL, 2.100 mmol) in THF (8 mL) and MeOH (2 mL) was stirred at room temperature for 2 h. Volatile materials were removed in vacuo. The residue was diluted with water acidified with 1 M HCl and extracted with EtOAc. The EtOAc extract was washed with water, dried (Na₂SO₄) and concentrated. The residue was triturated with hexanes:Et₂O (1:1) to give the title compound as a white solid.

¹H NMR (500 MHz, acetone-d₆): δ 8.08 (d, 2H), 7.81-7.76 (m, 1 μl), 7.67 (s, 1H), 7.18 (d, 2H), 7.08-7.01 (m, 2H), 5.85 (s, 2H). MS (+ESI) m/z 460, 462 (MH⁺).

### Example 2

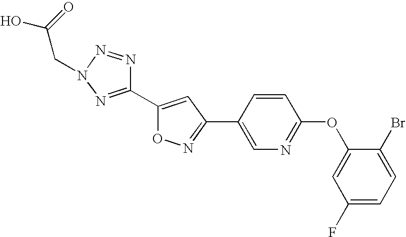

### (5-{3-[6-(2-Bromo-5-fluorophenoxy)pyridin-3-yl]isoxazol-5-yl}-2H-tetrazol-2-yl)acetic acid

The title compound was prepared in a similar manner as described in Example 1, steps 1 to 9, from 2-chloropyridine-5-carboxaldehyde and 2-bromo-5-fluorophenol.

¹H NMR (500 MHz, acetone-d₆): δ 8.77 (d, 1H), 8.53 (dd, 1H), 7.80 (dd, 1H), 7.76 (s, 1H), 7.34 (d, 1H), 7.30 (dd, 1H), 7.13 (td, 1H), 5.88 (s, 2H); MS (+ESI): m/z 461, 463 (MH⁺).

### Example 3

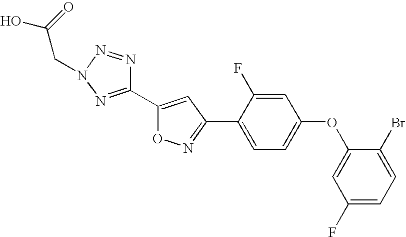

### (5-{3-[4-(2-Bromo-5-fluorophenoxy)-2-fluorophenyl]isoxazol-5-yl}-2H-tetrazol-2-yl)acetic acid

The title compound was prepared in a similar manner as described in Example 1, steps 1 to 9, from 2,4-difluorobenzaldehyde and 2-bromo-5-fluorophenol.

¹H NMR (300 MHz, DMSO-d₆): δ 8.05 (t, 1H), 7.85 (dd, 1H), 7.70 (d, 1H), 7.35 (dd, 1H), 7.20 (m, 2H), 6.98 (dd, 1H), 5.90 (s, 2H). MS: m/z 478, 480 (MH⁺).

### Example 4

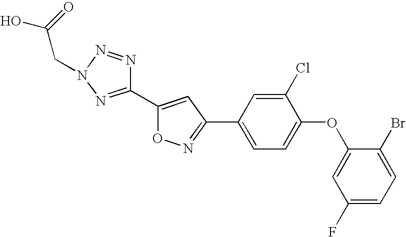

### (5-{3-[4-(2-Bromo-5-fluorophenoxy)-3-chlorophenyl]isoxazol-5-yl}-2H-tetrazol-2-yl)acetic acid

The title compound was prepared in a similar manner as described in Example 1, steps 1 to 9, from 3-chloro-4-fluorobenzaldehyde and 2-bromo-5-fluorophenol.

¹H NMR (300 MHz, DMSO-d₆): δ 8.30 (s, 1H), 8.05 (1H), 8.00 (d, 1H), 7.82 (t, 1H), 7.12 (m, 3H), 5.78 (s, 2H). MS: m/z 494, 496 (MH+).

### Example 5

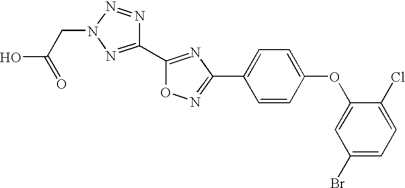

### (5-{3-[4-(5-Bromo-2-chlorophenoxy)phenyl]-1,2,4-oxadiazol-5-yl}-2H-tetrazol-2-yl)acetic acid

### Step 1: 4-(5-Bromo-2-chlorophenoxy)benzonitrile

To a solution of 5-bromo-2-chlorophenol (1.884 g, 9.08 mmol) and 4-fluorobenzonitrile (1 g, 8.26 mmol) in DMF (27.5 mL) was added potassium carbonate (2.282 g, 16.51 mmol). The reaction mixture was heated at 150° C. for 18 h. The mixture was cooled to RT, diluted with water (100 mL) and extracted with Et₂O (3×25 mL). The combined organic extracts were washed with 1 N NaOH (50 mL) then dried over Na₂SO₄. The product was recrystallized from Et₂O/hexanes, filtered and washed with hexanes to afford the title compound.

¹H NMR (500 MHz, acetone-d₆): δ 7.84 (d, 2H), 7.60 (d, 1H), 7.57-7.53 (m, 2H), 7.17 (d, 2H). MS: m/z 308, 310 (MH⁺).

### Step 2: 4-(5-Bromo-2-chlorophenoxy)-N′-hydroxybenzenecarboximidamide

To a mixture of 4-(5-bromo-2-chlorophenoxy)benzonitrile (0.5 g, 1.620 mmol) and hydroxylamine hydrochloride (0.135 g, 1.945 mmol) in EtOH (5.40 mL) was added triethylamine (0.339 mL, 2.431 mmol). The mixture was stirred at RT for 0.5 h and then heated at 60° C. for 1 h. The solvent was evaporated. The residue was diluted with water (10 mL) and extracted with EtOAc (3×10 mL). The combined organic extracts were dried over Na₂SO₄ and the solvent was evaporated under reduced pressure. The product was dissolved in a minimum amount of CH₂Cl₂ and precipitated with hexanes. The solid was filtered, washed with hexanes and dried under high vacuum to afford the title compound.

¹H NMR (500 MHz, acetone-d₆): δ 8.92 (s, 1H), 7.79 (d, 2H), 7.55 (d, 1H), 7.42 (dd, 1H), 7.27 (d, 1H), 7.05 (d, 2H), 5.50 (s, 1H). MS: m/z 341, 343 (MH⁺).

### Step 3: 3-[4-(5-Bromo-2-chlorophenoxy)phenyl]-1,2,4-oxadiazole-5-carboxamide

To a solution of 4-(5-bromo-2-chlorophenoxy)-N′-hydroxybenzenecarboximidamide (600 mg, 1.757 mmol) and pyridine (426 μL, 5.27 mmol) in THF (5.8 mL) was added methyl oxalyl chloride (359 μL, 3.86 mmol) at 0° C. The mixture was warmed to RT and stirred for 1 h. The solvent was evaporated and the residue was diluted with 1 N HCl (10 mL). The aqueous layer was extracted with EtOAc (3×10 mL) and dried over Na₂SO₄. The solvent was evaporated and the mixture was placed in a thick glass-walled vessel and dissolved in MeOH (5.8 mL). Ammonia gas was bubbled through for 2 min. The vessel was capped and the mixture was heated at 50° C. for 1 h. The mixture was cooled to RT and diluted with ether (5 mL). The mixture was filtered, washed with water and Et₂O, and dried under high vacuum to afford the title compound.

¹H NMR (500 MHz, DMSO-d₆): δ 8.52 (s, 1H), 8.47 (s, 1H), 8.08 (d, 2H), 7.65 (d, 1H), 7.57-7.52 (m, 2H), 7.19 (d, 2H). LCMS: m/z 418, 416 (MNa⁺).

### Step 4: 3-[4-(5-Bromo-2-chlorophenoxy)phenyl]-1,2,4-oxadiazole-5-carbonitrile

To a solution of 3-[4-(5-bromo-2-chlorophenoxy)phenyl]-1,2,4-oxadiazole-5-carboxamide (650 mg, 1.647 mmol) and triethylamine (1.8 mL, 13.18 mmol) in THF (5.5 mL) was added TFAA (814 μL, 5.77 mmol) at 0° C. and the mixture was stirred for 10 min. The solvent was evaporated and the residue was diluted with water (10 mL). The aqueous layer was extracted with EtOAc (3×10 mL). The combined organic extracts were dried over Na₂SO₄ and the solvent was evaporated. Purification by Combiflash chromatography (SiO₂-40 g, gradient elution of 0-10% EtOAc/hexanes over 25 min) afforded the title compound.

¹H NMR (500 MHz, acetone-d₆): δ 8.17 (d, 2H), 7.61 (d, 1H), 7.56-7.50 (m, 2H), 7.24 (d, 2

H).

### Step 5: 5-{3-[4-(5-Bromo-2-chlorophenoxy)phenyl]-1,2,4-oxadiazol-5-yl}-1H-tetrazole

A mixture of 3-[4-(5-bromo-2-chlorophenoxy)phenyl]-1,2,4-oxadiazole-5-carbonitrile (240 mg, 0.637 mmol), sodium azide (83 mg, 1.275 mmol) and ammonium chloride (102 mg, 1.912 mmol) in DMF (1.3 mL) was heated at 100° C. for 1 h. The mixture was cooled to RT, diluted with 1 N NaOH (1 mL) and washed with Et₂O (2×3 mL). The aqueous layer was acidified to pH about 1 with 2 N HCl and extracted with EtOAc (3×3 mL). The combined organic extracts were washed with water (3 mL) and then dried over Na₂SO₄. The solvent was evaporated under reduced pressure to afford the title compound.

¹H NMR (500 MHz, acetone-d₆): δ 8.25 (d, 2H), 7.99 (s, 1H), 7.61 (d, 1H), 7.54-7.49 (m, 2H), 7.25 (d, 2H). LCMS: m/z 419, 421 (MH⁺).

### Step 6: Ethyl (5-{3-[4-(5-bromo-2-chlorophenoxy)phenyl]-1,2,4-oxadiazol-5-yl}-2H-tetrazol-2-yl)acetate

A mixture of 5-{3-[4-(5-bromo-2-chlorophenoxy)phenyl]-1,2,4-oxadiazol-5-yl}-1H-tetrazole (210 mg, 0.500 mmol), triethylamine (140 μL, 1.0 mmol) and ethyl bromoacetate (83 μL, 0.751 mmol) in THF (1 mL) was heated at 70° C. for 1 h. The solvent was evaporated, the mixture was diluted with water (3 mL) and slurried with Et₂O (3 mL). The mixture was filtered and washed with water followed by Et₂O. The solid was dried under high vacuum to afford the title compound.

¹H NMR (500 MHz, acetone-d₆): δ 8.27-8.22 (m, 2H), 7.61 (d, 1H), 7.54-7.49 (m, 2H), 7.26-7.21 (m, 2H), 5.99 (d, 2H), 4.33 (q, 2H), 1.32 (t, 3H). MS: m/z 505, 5071 (MH⁺).

### Step 7: (5-{3-[4-(5-Bromo-2-chlorophenoxy)phenyl]-1,2,4-oxadiazol-5-yl}-2H-tetrazol-2-yl)acetic acid

To a solution of ethyl (5-{3-[4-(5-bromo-2-chlorophenoxy)phenyl]-1,2,4-oxadiazol-5-yl}-2H-tetrazol-2-yl)acetate (140 mg, 0.277 mmol) in THF (923 μL) and MeOH (461 μL) was added 2 N NaOH (277 μL, 0.554 mmol) and the mixture was stirred at RT for 10 min. The THF and MeOH were evaporated and the aqueous layer was washed with Et₂O (2×2 mL). The aqueous layer was acidified to pH 1 with 2 N HCl and stirred for 5 min. The mixture was filtered and washed with water followed by 1:1 Et₂O/hexanes. The solid was dried under high vacuum to afford the title compound.

¹H NMR (500 MHz, acetone-d₆): δ 8.26 (d, 2H), 7.61 (d, 1H), 7.53-7.49 (m, 2H), 7.25 (d, 2H), 5.96 (s, 2H). MS: m/z 477, 479 (MH⁺).

### Example 6

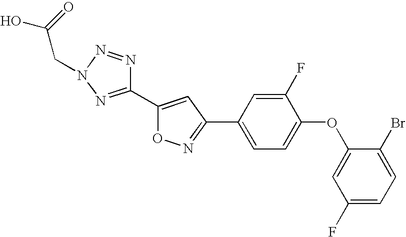

### (5-{3-[4-(2-Bromo-5-fluorophenoxy)-3-fluorophenyl]isoxazol-5-yl}-2H-tetrazol-2-yl)acetic acid

The title compound was prepared in a similar manner as described in Example 1, steps 1 to 9, from 3,4-difluorobenzaldehyde and 2-bromo-5-fluorophenol.

¹H NMR (300 MHz, DMSO-d₆): δ 8.05 (t, 1H), 7.85 (dd, 1H), 7.70 (d, 1H), 7.35 (dd, 1H), 7.20 (m, 2H), 6.98 (dd, 1H), 5.90 (s, 2H). MS: m/z 478, 480 (MH⁺).

### Example 7

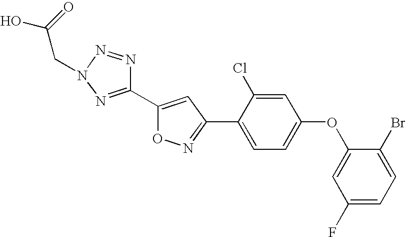

### (5-{3-[4-(2-Bromo-5-fluorophenoxy)-2-chlorophenyl]isoxazol-5-yl}-2H-tetrazol-2-yl)acetic acid

The title compound was prepared in a similar manner as described in Example 1, steps 1 to 9, from 2-chloro-4-fluorobenzaldehyde and 2-bromo-5-fluorophenol.

¹H NMR (300 MHz, DMSO-d₆): δ 8.28 (d, J=2 Hz, 1H), 8.03 (s, 1H), 7.99 (dd, J=2 Hz and 9 Hz, 1H), 7.81-7.85 (m, 1H), 7.12 (t, J=8 Hz, 3H), 5.76 (s, 2H). MS: m/z 494, 496 (MH⁺).

### Example 8

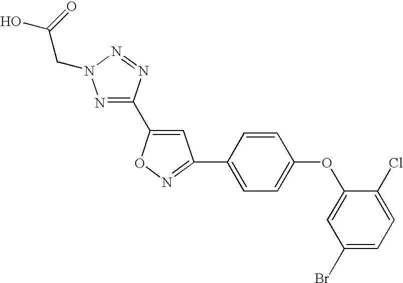

### (5-{3-[4-(5-Bromo-2-chlorophenoxy)phenyl]isoxazol-5-yl}-2H-tetrazol-2-yl)acetic acid

The title compound was prepared in a similar manner as described in Example 1, steps 1 to 9, from 4-fluorobenzaldehyde and 5-bromo-2-chlorophenol.

¹H NMR (400 MHz, DMSO-d₆): δ 8.05 (d, J=9 Hz, 2H), 7.95 (s, 1H), 7.61 (d, J=9 Hz, 1H), 7.46-7.51 (m, 2H), 7.14 (d, J=9 Hz, 2H), 5.86 (s, 2H). MS: m/z 476, 478 (MH⁺).

### Example 9

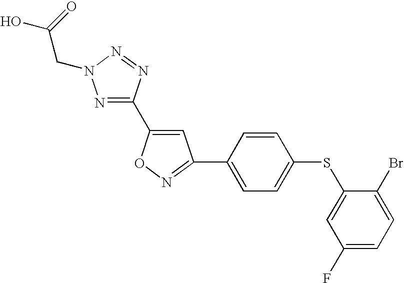

### [5-(3-{4-[(2-Bromo-5-fluorophenyl)thio]phenyl}isoxazol-5-yl)-2H-tetrazol-2-yl]acetic acid

The title compound was prepared in a similar manner as described in Example 1, steps 1 to 9, from 4-fluorobenzaldehyde and 2-bromo-5-fluorothiophenol.

¹H NMR (400 MHz, DMSO-d₆): δ 8.09 (d, J=9 Hz, 2H), 7.99 (s, 1H), 7.75 (dd, J=5 Hz and 9 Hz, 1H), 7.58 (d, J=9 Hz, 2H), 7.09-7.13 (m, 1H), 6.86 (dd, J=3 Hz and 9 Hz, 1H), 5.86 (s, 2H). MS: m/z 476 and 478 (MH⁺).

### Example 10

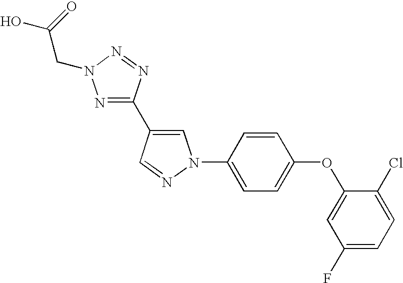

### (5-{1-[4-(2-Chloro-5-fluorophenoxy)phenyl]-1H-pyrazol-4-yl}-2H-tetrazol-2-yl)acetic acid

### Step 1: 1-Chloro-4-fluoro-2-(4-nitrophenoxy)benzene

A mixture of 1-fluoro-4-nitrobenzene (8.6 g, 60.9 mmol), 2-chloro-5-fluorophenol (9.38 g, 64.0 mmol) and potassium carbonate (16.85 g, 122 mmol) in DMF (100 mL) was heated at 100° C. overnight. After cooling, the mixture was diluted with water and extracted with EtOAc. The EtOAc extract was washed with diluted brine (2×), dried (Na₂SO₄) and concentrated. Chromatography over silica gel and elution with hexanes:EtOAc (9:1) gave the title compound as a pale yellow oil, which solidified in contact with small amount EtOH.

¹H NMR (500 MHz, acetone-d₆): δ 8.36-8.28 (m, 2H), 7.70 (dd, 1H), 7.26-7.17 (m, 4H).

### Step 2: 1-Chloro-4-fluoro-2-(4-iodophenoxy)benzene

To a mixture of 1-chloro-4-fluoro-2-(4-nitrophenoxy)benzene (13 g, 41.7 mmol), ammonium chloride (1.2 g, 22.43 mmol) and iron powder (12 g, 215 mmol) in ethanol (200 mL) and water (100 mL) was heated at reflux temperature for 1 h. The hot mixture was filtered through celite and the filtered cake was washed with EtOH. The combined filtrates were concentrated in vacuo to remove volatile materials. The residue was diluted with water and extracted with EtOAc. The EtOAc extract was washed with water, dried (Na₂SO₄) and concentrated to give the 4-(2-chloro-5-fluorophenoxy)aniline as a pale yellow oil.

To a suspension of 4-(2-chloro-5-fluorophenoxy)aniline (10 g, 42.1 mmol) in water (35 mL) was added 6 M hydrochloric acid (35.1 mL, 210 mmol). A white precipitate formed and the mixture was cooled with an ice-water bath. A small amount of acetone was used to washed down the solid on the side of the reaction flask to the main mixture. After about 5-10 min, a solution of 4M sodium nitrite (12 mL, 48.0 mmol) was added dropwise over 10-15 min. The mixture was further stirred for 1 h at 0° C. A solution of 6 M potassium iodide (14 g, 84 mmol) was added over about 15 min. At one point, EtOAc was added to break up the solid to facilitate the stirring. After further stirring for 15 min, the mixture was diluted with water and extracted with EtOAc. The EtOAc layer was separated, washed with Na₂SO₃ and water, dried (Na₂SO₄) and concentrated. Chromatography over silica gel and elution with hexanes:EtOAc (5:1) gave the title compound as a light brown liquid.

¹H NMR (500 MHz, acetone-d₆): δ 7.80-7.75 (m, 2H), 7.62 (dd, 1H), 7.12-7.03 (m, 1H), 7.02-6.96 (m, 1H), 6.92-6.87 (m, 2H).

### Step 3: Ethyl 1-[4-(2-chloro-5-fluorophenoxy)phenyl]-1H-pyrazole-4-carboxylate

To a solution of 1-chloro-4-fluoro-2-(4-iodophenoxy)benzene (2.98 g, 8.56 mmol) in toluene (15 mL) was added ethyl 1H-pyrazole-4-carboxylate (1 g, 7.14 mmol), potassium carbonate (2.071 g, 14.99 mmol), copper(I) iodide (0.068 g, 0.357 mmol) and rac-trans-N,N′-dimethylcyclohexane-1,2-diamine (0.203 g, 1.427 mmol). The mixture was then purged with N₂ for 15-20 min and heated at 110° C. overnight. After cooling, the whole mixture was filtered through silica and washed with hexanes-EtOAc (1:1). The filtrate was concentrated. The residue was purified by Combi-Flash™ chromatography (120 g, 15-30% EtOAc in hexanes for 20 min, 75 mL/min, 25 mL/fraction). Trituration with hexanes:Et2O (1:1) gave the title compound as a white solid.

¹H NMR (500 MHz, acetone-d₆): δ 8.82 (s, 1H), 8.08 (s, 1H), 8.02-7.97 (m, 2H), 7.64 (dd, 1H), 7.25-7.20 (m, 2H), 7.09 (td, 1H), 7.01 (dd, 1H), 4.31 (q, 2H), 1.35 (t, 3H). MS (+ESI): m/z 361 (MH⁺).

### Step 4: 1-[4-(2-Chloro-5-fluorophenoxy)phenyl]-1H-pyrazole-4-carboxamide

A mixture of ethyl 1-[4-(2-chloro-5-fluorophenoxy)phenyl]-1H-pyrazole-4-carboxylate (2.5 g, 6.93 mmol) and a solution of 1M NaOH (14 mL, 14.00 mmol) in THF (30 mL) and MeOH (15 mL) was heated at 70° C. for 2 h. Volatile materials were removed in vacuo. The residue was diluted with water and acidified with 1M HCl. The precipitate formed was collected, washed with water and dried under vacuum to give the 1-[4-(2-chloro-5-fluorophenoxy)phenyl]-1H-pyrazole-4-carboxylic acid as a white powder.

To a suspension of 1-[4-(2-chloro-5-fluorophenoxy)phenyl]-1H-pyrazole-4-carboxylic acid (2 g, 6.01 mmol) and a drop of DMF in CH₂Cl₂ (10 mL) and THF (40 mL) was added oxalyl chloride (1.1 mL, 12.57 mmol). The mixture became homogeneous after about 5 min and was further stirred for 1 h. Volatile materials were removed in vacuo. The residue was re-dissolved in CH₂Cl₂ and evaporated again (2×), dried under vacuum to give the crude 1-[4-(2-chloro-5-fluorophenoxy)phenyl]-1H-pyrazole-4-carbonyl chloride as a pale yellow solid which was used for next reaction without further purification.

The crude 1-[4-(2-chloro-5-fluorophenoxy)phenyl]-1H-pyrazole-4-carbonyl chloride (2.1 g, 5.98 mmol) was dissolved in THF (40 mL). The mixture was cooled to 0° C. and then ammonia gas was bubbled into the solution surface for about 1 to 2 min. The mixture was turned cloudy and was further stirred for 10 min. Volatile materials were removed in vacuo. The residue was suspended in water and filtered. The white solid was washed with ether and dried under vacuum to give the title compound.

¹H NMR (400 MHz, acetone-d₆): δ 8.75 (s, 1H), 8.12 (s, 1H), 7.97-7.92 (m, 2H), 7.65 (dd, 1H), 7.26-7.21 (m, 2H), 7.08 (ddd, 1H), 7.00 (dd, 1H), 6.49 (s, 1H). MS (+ESI): m/z 332 (MH⁺).

### Step 5: (5-{1-[4-(2-Chloro-5-fluorophenoxy)phenyl]-1H-pyrazol-4-yl}-2H-tetrazol-2-yl)acetic acid

The title compound was prepared in a similar manner as described for Example 1, step 6 to 9, from 1-[4-(2-chloro-5-fluorophenoxy)phenyl]-1H-pyrazole-4-carboxamide.

¹H NMR (500 MHz, acetone-d₆): δ 8.94 (s, 1H), 8.25 (s, 1H), 8.09-8.03 (m, 2H), 7.65 (dd, 1H), 7.28-7.22 (m, 2H), 7.08 (td, 1H), 7.01 (dd, 1H), 5.73 (s, 2H). MS: m/z 415 (MH⁺).

### Example 11

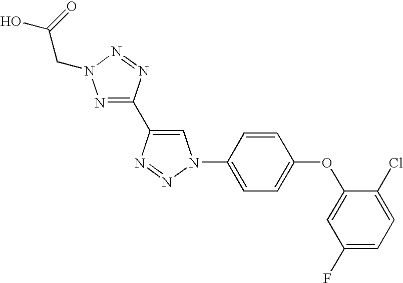

### (5-{1-[4-(2-Chloro-5-fluorophenoxy)phenyl]-1H-1,2,3-triazol-4-yl}-2H-tetrazol-2-yl)acetic acid

### Step 1: 2-(4-Azidophenoxy)-1-chloro-4-fluorobenzene

To a stirred solution of 4-(2-chloro-5-fluorophenoxy)aniline (238 mg, 1.0 mmol) in 5 mL of HCl (6 M) was added a solution of 4M NaNO₂ (69 mg, 1.0 mmol) with cooling in an ice-bath. The reaction mixture was stirred for 20 min at 0-5° C. Sodium azide (78 mg, 1.2 mmol) was added and the mixture was stirred at room temperature for 2 h. The reaction was worked up by dilution with EtOAc (100 mL). The organic layer was washed with brine (50 mL) and dried over Na₂SO₄. After evaporated, the crude was purified by preparative TLC (PE/EA=10/1) to afford the title compound.

¹H NMR (CDCl₃, 400 MHz): δ 7.40 (dd, 1H), 6.99-7.05 (m, 4H), 6.70-6.75 (m, 1H). 6.62 (dd, 1H).

### Step 2: Ethyl 1-[4-(2-chloro-5-fluorophenoxy)phenyl]-1H-1,2,3-triazole-4-carboxylate

To a stirred solution of 2-(4-azidophenoxy)-1-chloro-4-fluorobenzene (2.37 g, 9.0 mmol) in 30 mL of toluene was added ethyl propiolate (2.7 mL, 27.0 mmol). After stirring overnight, the solvent was removed in vacuum. The residue was purified by silica gel column (PE/EA=8/1) to afford the title compound.

¹H NMR (CDCl₃, 400 MHz): δ 8.45 (s, 1H), 7.72 (d, 2H), 7.44 (dd, 1H), 7.11 (d, 2H), 6.87-6.92 (m, 1H), 6.79 (dd, 1H), 4.45 (q, 2H), 1.42 (t, 3H). MS: m/z 362 (MH⁺).

### Step 3: (5-{1-[4-(2-Chloro-5-fluorophenoxy)phenyl]-1H-1,2,3-triazol-4-yl}-2H-tetrazol-2-yl)acetic acid

The title compound was prepared in a similar manner as described for Example 10, step 4 and 5, from ethyl 1-[4-(2-chloro-5-fluorophenoxy)phenyl]-1H-1,2,3-triazole-4-carboxylate.

¹H NMR (400 MHz, DMSO-d₆): δ 13.80 (s, 1H), 9.56 (s, 1H), 8.05 (d, J=9 Hz, 2H), 7.71 (dd, J=6 Hz and 9 Hz, 1H), 7.17-7.26 (m, 4H), 5.80 (s, 2H). MS: m/z 416 (MH⁺).

### Example 12

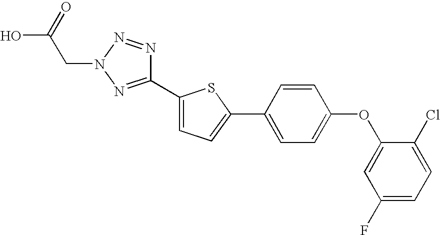

### (5-{5-[4-(2-Chloro-5-fluorophenoxy)phenyl]-2-thienyl}-2H-tetrazol-2-yl)acetic acid

### Step 1: 2-[4-(2-Chloro-5-fluorophenoxy)phenyl]-4,4,5,5-tetramethyl-1,3,2-dioxaborolane

To a mixture of 1-chloro-4-fluoro-2-(4-iodophenoxy)benzene (3.49 g, 10 mmol) from Example 10, step 2, bis(pinacolato)diboron (2.79 g, 11.00 mmol), potassium acetate (2.94 g, 30.0 mmol) and palladium(II) acetate (0.1 g, 0.445 mmol) in DMF (40 mL) was bubbled N₂ gas for 15-30 min. The mixture was then heated at 85° C. for 3 h. After cooling the mixture was diluted with water and extracted with EtOAc (2×). The EtOAc extracts were combined, washed with diluted brine, dried (Na₂SO₄) and concentrated. CombiFlash™ (120 g, 5-15% EtOAc in hexanes for 20 min, 75 mL/min, 25 mL/fraction) afforded the tile compound.

### Step 2: 5-[4-(2-Chloro-5-fluorophenoxy)phenyl]thiophene-2-carboxamide

To a solution of 5-bromothiophene-2-carboxamide (0.7 g, 3.40 mmol), 2-[4-(2-chloro-5-fluorophenoxy)phenyl]-4,4,5,5-tetramethyl-1,3,2-dioxaborolane (2.2 g, 3.79 mmol) from step 1 and 2M sodium carbonate (3.40 mL, 6.79 mmol) in DMF (30 mL) was bubbled N₂ gas for 15 min and then 1,1′-bis(diphenylphosphino)ferrocenedichloro palladium(II) dichloromethane complex (0.050 g, 0.068 mmol) was added. The mixture was heated at 80° C. for 3 h. After cooling, the mixture was diluted with water and extracted with EtOAc. The EtOAc extract was washed with water (2×), filtered through celite, dried (Na₂SO₄) and concentrated. The residue was dissolved in small amount of acetone, passed a short column of silica gel and eluted with EtOAc. Solvents were evaporated in vacuo. The resulting residue was triturated with Et₂O to give the title compound as a white solid.

¹H NMR (500 MHz, acetone-d₆): δ 7.79 (d, 2H), 7.73 (d, 1H), 7.64 (dd, 1H), 7.45 (m, 2H), 7.14-7.05 (m, 3H), 7.01 (dd, 1H), 6.70 (s, 1H). MS: m/z 348 (MH⁺).

### Step 3: (5-{5-[4-(2-Chloro-5-fluorophenoxy)phenyl]-2-thienyl}-2H-tetrazol-2-yl)acetic acid

The title compound was prepared in a similar manner as described for Example 1, step 6 to 9, from 5-[4-(2-chloro-5-fluorophenoxy)phenyl]thiophene-2-carboxamide.

¹H NMR (500 MHz, acetone-d₆): δ 7.86-7.82 (m, 3H), 7.64 (dd, 1H), 7.57 (d, 1H), 7.17-7.13 (m, 2H), 7.09 (ddd, 1H), 7.02 (dd, 1H), 5.75 (s, 2H). MS: m/z 431 (MH⁺).

### Example 13

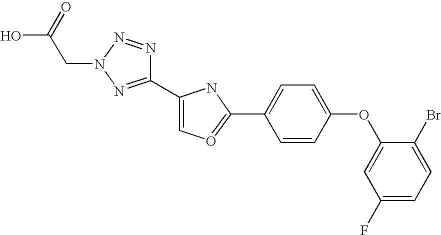

(5-{2-[4-(2-Bromo-5-fluorophenoxy)phenyl]-1,3-oxazol-4-yl}-2H-tetrazol-2-yl)acetic acid

### Step 1: Ethyl 4-(2-bromo-5-fluorophenoxy)benzoate

To a mixture of ethyl 4-fluorobenzoate (2.5 g, 14.87 mmol) and 2-bromo-5-fluorophenol (4.26 g, 22.30 mmol) in DMF (37.2 ml) was added potassium carbonate (3.08 g, 22.30 mmol). The reaction mixture was heated to 140° C. for 15 h. The mixture was diluted with water/HCl 1 N (100 mL) and extracted with EtOAc (3×25 mL). The combined organic extracts were washed with 1N HCl (50 mL), water (50 mL) and brine (50 mL), then dried over MgSO₄. The solvent was evaporated under reduced pressure and the residue was purified by Combiflash™ (SiO₂-120 g, elution with 0.5-1% acetone/hexanes over 30 min) to afford the title compound as an oil.

¹H NMR (500 MHz, acetone-d₆): δ 8.07-8.03 (m, 2H), 7.81-7.76 (m, 1H), 7.10-7.04 (m, 4H), 4.30 (q, 2H), 1.34 (t, 3H).

### Step 2: 4-(2-Bromo-5-fluorophenoxy)benzoic acid

The title compound was prepared in a similar manner as described in Example 1, step 9, from ethyl 4-(2-bromo-5-fluorophenoxy)benzoate and NaOH.

¹H NMR (500 MHz, acetone-d₆): δ 8.07 (d, 2H), 7.81-7.77 (m, 1H), 7.09-7.05 (m, 4H). MS: m/z 309, 311 (MH⁺).

### Step 3: Methyl 2-{[4-(2-bromo-5-fluorophenoxy)benzoyl]amino}-3-hydroxypropanoate

To a solution of 4-(2-bromo-5-fluorophenoxy)benzoic acid (510 mg, 1.639 mmol), methyl 2-amino-3-hydroxypropanoate hydrochloride (255 mg, 1.639 mmol) and HOBT (276 mg, 1.803 mmol) in DMF (4.1 mL) was added N-methylmorpholine (415 μL 3.77 mmol). The reaction mixture was cooled to 0° C. and EDC (346 mg, 1.803 mmol) was added. The reaction was stirred at RT for 15 h. The reaction mixture was diluted with EtOAc (30 mL) and then washed with HCl 1N (20 mL), followed by saturated aqueous NaHCO₃ (20 mL) and brine (20 mL). The organic layer was dried (MgSO₄), filtered and evaporated under reduced pressure. The residue was purified by Combiflash™ (SiO₂-40 g, eluting with 0-70% EtOAc/Hexanes over 50 min) to afford the title compound as a foamy solid.

¹H NMR (500 MHz, acetone-d₆): δ 7.99 (d, 2H), 7.78 (dd, 1H), 7.73 (d, 1H), 7.08-6.98 (m, 4 H), 4.74-4.72 (m, 1H), 4.30-4.27 (m, 1H), 3.99-3.93 (m, 2H), 3.71 (s, 3H).

### Step 4: Methyl 2-[4-(2-bromo-5-fluorophenoxy)phenyl]-4,5-dihydro-1,3-oxazole-4-carboxylate

To a solution of methyl 2-{[4-(2-bromo-5-fluorophenoxy)benzoyl]amino}-3-hydroxypropanoate (600 mg, 1.456 mmol) in acetonitrile (5.8 ml)/dichloromethane (1.4 mL) was added triphenylphosphine (573 mg, 2.18 mmol) and DIPEA (407 μL, 2.33 mmol) CCl₄ (211 μL, 2.183 mmol) was added slowly and the reaction mixture was stirred at RT for 8 h. The reaction mixture was cooled to 0° C. and EtOAc was added (25 mL), followed by saturated aqueous NaHCO₃ (50 mL). After stirring for 10 min, the organic layer was washed with brine (50 mL), dried (MgSO₄), filtered and evaporated under reduced pressure. The residue was purified by Combiflash™ (SiO₂-40 g, eluting with 0-40% EtOAc/Hexanes over 50 min) to afford the title compound.

¹H NMR (500 MHz, acetone-d₆): δ 7.98 (d, 2H), 7.78 (dd, 1H), 7.09-7.01 (m, 4H), 4.93 (t, 1H), 4.64 (d, 2H), 3.73 (s, 3H). MS (+ESI) m/z 395, 397 (MH⁺).

### Step 5: Methyl 2-[4-(2-bromo-5-fluorophenoxy)phenyl]-1,3-oxazole-4-carboxylate

To a solution of copper (II) bromide (1.05 g, 4.72 mmol), hexamethylenetetramine (661 mg, 4.72 mmol) and DBU (711 μL, 4.72 mmol) in DCM (10 mL) was added methyl 2-[4-(2-bromo-5-fluorophenoxy)phenyl]-4,5-dihydro-1,3-oxazole-4-carboxylate (465 mg, 1.180 mmol). The reaction mixture was stirred at RT for 5 h. The solvent was evaporated under reduced pressure. The residue was diluted with EtOAc (25 mL) and saturated NH₄Cl/NH₄OH (1:1) (50 mL) and extracted with EtOAc (2×25 mL). The combined organic layers were washed with sat NH₄Cl/NH₄OH (1:1) (25 mL), 5% citric acid in water (25 ml), NaHCO₃ (25 mL) and brine (25 mL), then dried (MgSO₄), filtered and evaporated under reduced pressure. The residue was purified by Combiflash™ chromatography (SiO₂-12 g, eluting with 0-40% EtOAc/Hexanes over 20 min) to afford the title compound as a solid.

¹H NMR (500 MHz, acetone-d₆): δ 8.66 (s, 1H), 8.14 (d, 2H), 7.83 (dd, 1H), 7.20 (d, 2H), 7.13-7.09 (m, 2H), 3.90 (s, 3H). MS (+ESI) m/z 392, 394 (MH⁺).

### Step 6: 2-[4-(2-Bromo-5-fluorophenoxy)phenyl]-1,3-oxazole-4-carboxamide

In a sealed tube, methyl 2-[4-(2-bromo-5-fluorophenoxy)phenyl]-1,3-oxazole-4-carboxylate (150 mg, 0.369 mmol) was dissolved in MeOH (5 mL) and the reaction mixture was cooled down to 0° C. Ammonia gas was bubbled into solution for 5 min. The reaction mixture was then stirred at 60° C. for 18 h. Volatile materials were removed in vacuo and the residue was triturated in DCM/Hexanes (1:1) to afford the title compound as a white solid.

¹H NMR (500 MHz, acetone-d₆): δ 8.45 (s, 1H), 8.12 (d, 2H), 7.84-7.79 (m, 1H), 7.33 (s, 1H), 7.21-7.18 (m, 2H), 7.10-7.07 (m, 2H), 6.81 (s, 1H) MS (+ESI) m/z 377, 379 (MH⁺).

### Step 7: (5-{2-[4-(2-Bromo-5-fluorophenoxy)phenyl]-1,3-oxazol-4-yl}-2H-tetrazol-2-yl)acetic acid

The title compound was prepared in a similar manner as described for Example 1, step 6 to 9, from 2-[4-(2-bromo-5-fluorophenoxy)phenyl]-1,3-oxazole-4-carboxamide.

¹H NMR (500 MHz, acetone-d₆): δ 8.65 (s, 1H), 8.19 (d, 2H), 7.84-7.81 (m, 1H), 7.21 (d, 2H), 7.10 (d, 2H), 5.30 (s, 2H) MS: m/z 460, 462 (MH⁺).

### Example of a Pharmaceutical Formulation

As a specific embodiment of an oral pharmaceutical composition of the present invention, a 100 mg potency tablet is composed of 100 mg of any one of Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I:
HetAr—W—X—Ar (I)
or a pharmaceutically acceptable salt thereof; wherein
X is —O—, —S—, —S(O)—, —S(O)₂—, —NR⁹—, or —CR¹⁰R¹¹—;
W is selected from the group consisting of: 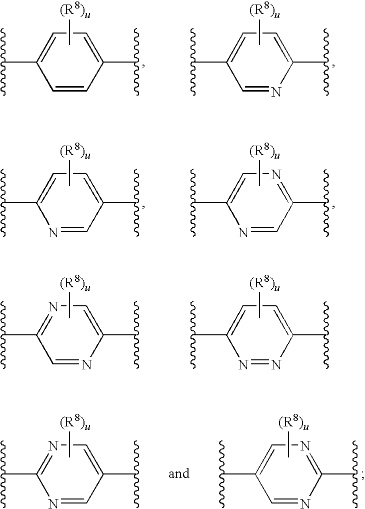
HetAr is heteroaryl selected from the group consisting of: 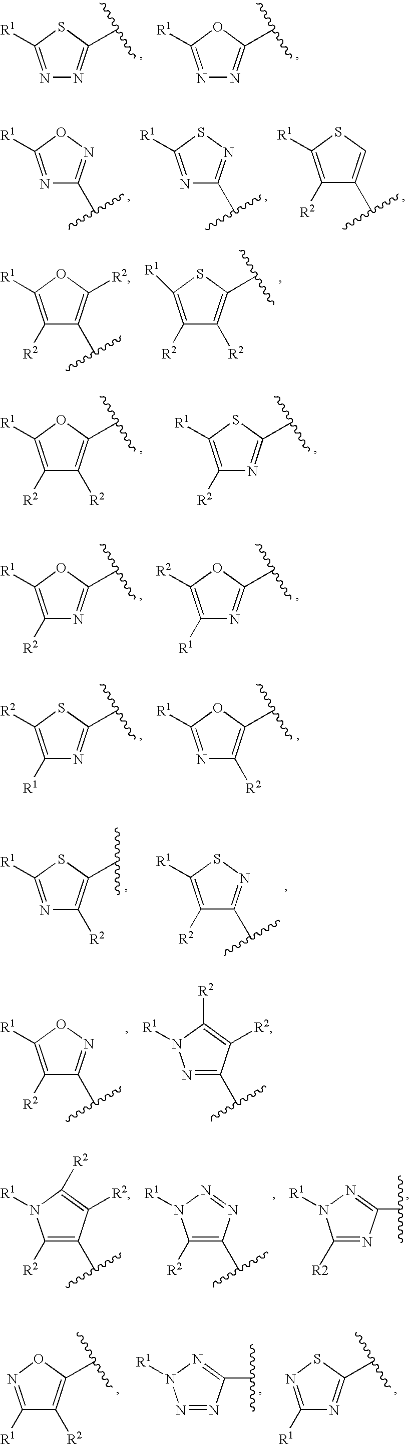 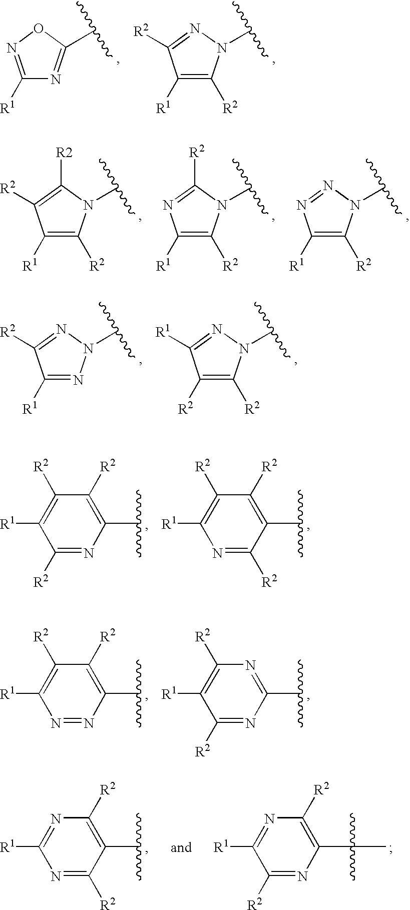
R¹ is heteroaryl selected from the group consisting of: 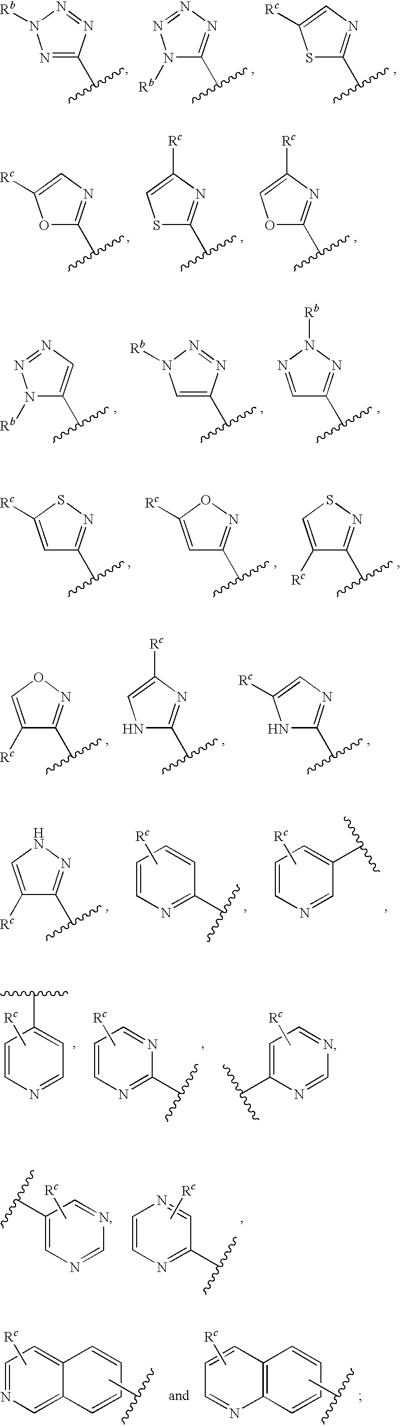
wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;
and wherein said R¹ heteroaryl ring is optionally substituted with a substituent selected from the group consisting of cyano, halogen, C₁₋₄ alkyl, C₁₋₄ alkoxy, C₁₋₄ alkylthio, C₁₋₄ alkylsulfonyl, and trifluoromethyl;
each R² is independently selected from the group consisting of:
hydrogen,
halogen,
hydroxy,
cyano,
amino,
nitro,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl,
carboxy,
alkyloxycarbonyl, and
C₁₋₄ alkylcarbonyl;
Ar is phenyl or naphthyl optionally substituted with one to five R³ substituents;
each R³ is independently selected from the group consisting of:
C₁₋₆ alkyl,
C₁₋₆ alkenyl,
(CH₂)ₙ-phenyl,
(CH₂)ₙ-naphthyl,
(CH₂)ₙ-heteroaryl,
(CH₂)ₙ-heterocyclyl,
(CH₂)ₙC₃₋₇ cycloalkyl,
halogen,
nitro,
(CH₂)ₙOR⁴,
(CH₂)ₙN(R⁴)₂,
(CH₂)ₙC≡N,
(CH₂)ₙCO₂R⁴,
(CH₂)ₙNR⁴SO₂R⁴
(CH₂)ₙSO₂N(R⁴)₂,
(CH₂)ₙS(O)₀₋₂R⁴,
(CH₂)ₙNR⁴C(O)N(R⁴)₂,
(CH₂)ₙC(O)N(R⁴)₂,
(CH₂)ₙNR⁴C(O)R⁴,
(CH₂)ₙNR⁴CO₂R⁴,
(CH₂)ₙC(O)R⁴,
(CH₂)ₙC(O)N(R⁴)₂,
(CH₂)ₛ—Z—(CH₂)ₜ-phenyl,
(CH₂)ₛ—Z—(CH₂)ₜ-naphthyl,
(CH₂)ₛ—Z—(CH₂)ₜ-heteroaryl,
(CH₂)ₛ—Z—(CH₂)ₜ-heterocyclyl,
(CH₂)ₛ—Z—(CH₂)ₜ—C₃₋₇ cycloalkyl,
(CH₂)ₛ—Z—(CH₂)ₜ—OR⁴,
(CH₂)ₛ—Z—(CH₂)ₜ—N(R⁴)₂,
(CH₂)ₛ—Z—(CH₂)ₜ—NR⁴SO₂R⁴,
(CH₂)ₛ—Z—(CH₂)ₜ—C≡N,
(CH₂)ₛ—Z—(CH₂)ₜ—CO₂R⁴,
(CH₂)ₛ—Z—(CH₂)ₜ—SO₂N(R⁴)₂,
(CH₂)ₛ—Z—(CH₂)ₜ—S(O)₀₋₂R⁴,
(CH₂)ₛ—Z—(CH₂)ₜ—NR⁴C(O)N(R⁴)₂,
(CH₂)ₛ—Z—(CH₂)ₜ—C(O)N(R⁴)₂,
(CH₂)ₛ—Z—(CH₂)ₜ—NR⁴C(O)R⁴,
(CH₂)ₛ—Z—(CH₂)ₜ—NR⁴CO₂R⁴,
(CH₂)ₛ—Z—(CH₂)ₜ—C(O)R⁴,
CF₃,
CH₂CF₃,
OCF₃, and
OCH₂CF₃;
in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
Z is O, S, or NR⁴;
each R⁴ is independently selected from the group consisting of
hydrogen,
C₁₋₆ alkyl,
(CH₂)ₙ-phenyl,
(CH₂)ₙ-heteroaryl,
(CH₂)ₙ-naphthyl, and
(CH₂)ₙC₃₋₇ cycloalkyl;
wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
each R⁶ and R⁷ are independently hydrogen or C₁₋₃ alkyl, wherein alkyl is optionally substituted with one to five fluorines;
each R⁸ is independently selected from the group consisting of hydrogen, halogen, and C₁₋₄ alkyl wherein alkyl is optionally substituted with one to five fluorines;
R⁹, R¹⁰, and R¹¹ are each independently hydrogen or C₁₋₃ alkyl, wherein alkyl is optionally substituted with one to five fluorines;
u is an integer from 0 to 2;
r is an integer from 0 to 3;
m is an integer from 1 to 3;
each p is independently an integer from 1 to 3;
each n is independently an integer from 0 to 2;
each s is independently an integer from 1 to 3; and
each t is independently an integer from 1 to 3.

2. The compound of claim 1 wherein X is —O—.

3. The compound of claim 1 wherein Ar is phenyl substituted with one to three R³ substituents.

4. The compound of claim 1 wherein W is phenyl or pyridyl wherein phenyl and pyridyl are optionally substituted with one or two R⁸ substituents.

5. The compound of claim 4 wherein W is unsubstituted phenyl.

6. The compound of claim 1 wherein HetAr is heteroaryl selected from the group consisting of:

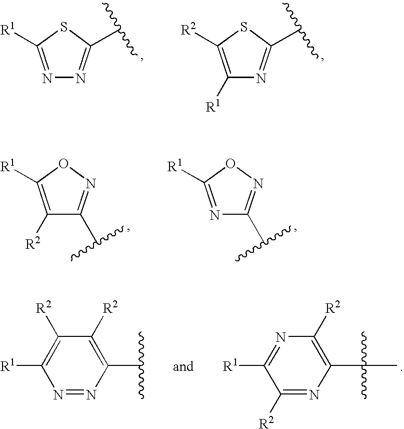

7. The compound of claim 6 wherein R² is hydrogen.

8. The compound of claim 6 wherein Het Ar is

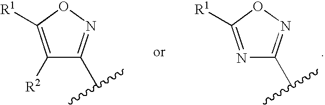

9. The compound of claim 8 wherein R² is hydrogen.

10. The compound of claim 1 wherein R¹ is heteroaryl selected from the group consisting of: 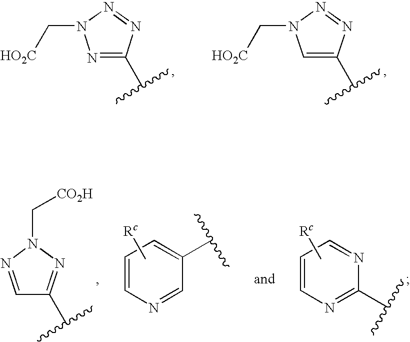
wherein R^{c} is —CO₂H, —CO₂C₁₋₃ alkyl, —CH₂CO₂H, or —CH₂CO₂C₁₋₃ alkyl.

11. The compound of claim 10 wherein R¹ is

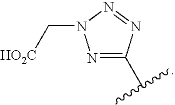

12. The compound of claim 1 wherein HetAr is heteroaryl selected from the group consisting of: 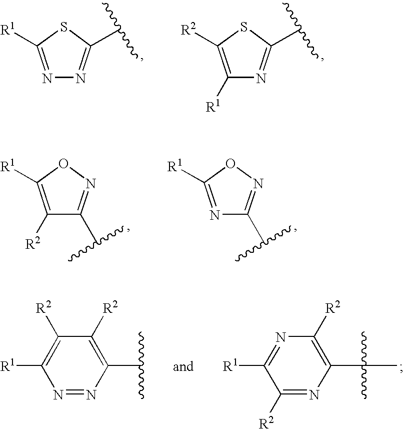
and R¹ is heteroaryl selected from the group consisting of: 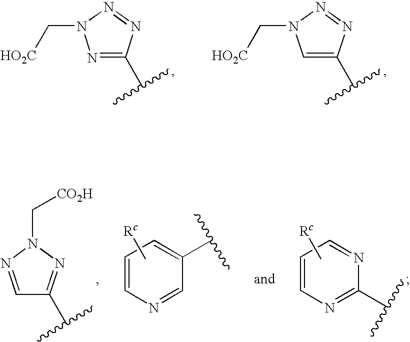
wherein R^{c} is —CO₂H, —CO₂C₁₋₃ alkyl, —CH₂CO₂H, or —CH₂CO₂C₁₋₃ alkyl.

13. The compound of claim 12 wherein HetAr is 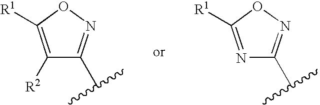
and R¹ is

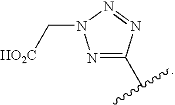

14. A pharmaceutical composition comprising a compound in accordance with claim 1 in combination with a pharmaceutically acceptable carrier.

15-19. (canceled)

20. A method for treating non-insulin dependent (Type 2) diabetes, insulin resistance, hyperglycemia, a lipid disorder, obesity, and fatty liver disease in a mammal in need thereof which comprises the administration to the mammal of a therapeutically effective amount of a compound of claim 1.

21. The method of claim 21 wherein said lipid disorder is selected from the group consisting of dyslipidemia, hyperlipidemia, hypertriglyceridemia, atherosclerosis, hypercholesterolemia, low HDL, and high LDL.
